(19) United States
(12) Patent Application Publication
(10) Pub. No.: US 2026/0263262 A1
(43) Pub. Date: Sep. 10, 2026

(54) ADHESIVE MOUTH COVERING SLEEP AID AND TRAINING DEVICE

(71) Applicant: Seald Wellness LLC, San Clemente, CA (US)

(72) Inventors: Haley DUNCAN, San Clemente, CA (US); Kelsey GARBE, Rancho Mission Viejo, CA (US); Briauna BOSANKO, Mission Viejo, CA (US)

(21) Appl. No.: 19/073,971

(22) Filed: Mar. 7, 2025

(51) Int. Cl. A61F 5/56

(52) U.S. Cl. CPC A61F 5/56

(57) ABSTRACT

A mouth tape is provided that gently adheres to the skin within the mouth area of a wearer. The mouth tape includes a strip of covering material that is moderately elastic and breathable, and an adhesive layer formed of a medical-grade adhesive that is non-allergenic and does not irritate the skin for substantially all known skin types. The strip is “lip-shaped” in that it covers the lips but does not extend into the cheek, chin, or nasal areas. An aperture through the strip provides airflow as well as access to the oral cavity, e.g. for the passage of a straw. The covering material is non-allergenic and may be a cotton/spandex blend. The adhesive is non-water-soluble so that it maintains adhesion to skin that has been treated with common skincare products such as creams, lotions, night masks, and lip serums.

### FIELD OF INVENTION

The present invention relates to the field of external aids for correcting sleep dysfunctions, and more particularly to mouth coverings that reduce mouth breathing and snoring.

### BACKGROUND

The act of “mouth breathing,” or inhaling and exhaling through the mouth instead of—or in addition to—the nostrils, is well known to have adverse health impacts on both short-term well-being and long-term development of a person with mouth-breathing tendencies. In contrast to nasal breathing, mouth breathing does not produce nitric oxide, which helps dilate blood vessels; consequently, mouth breathing can reduce oxygen absorption in the body. Mouth breathing can also increase the body's exposure to pathogens and the risk of infections. Mouth breathing causes dry mouth and reduces saliva production, which can lead to tooth decay and periodontal disease. Mouth breathing can hinder development of the face and jaw, and cause speech impediments. Mouth breathing during sleep contributes to obstructive sleep apnea (OSA) and snoring, leading to poor sleep quality and daytime fatigue. Outcomes also include dysfunctional breathing patterns, which contribute to elevations in cortisol levels and consequently more stress and anxiety.

There are several proven approaches to combat mouth breathing tendencies, depending on the source of the dysfunction. Medical intervention may be required to correct developmental issues, nasal congestion, deviated septum, allergies, and similar causes. Exercise regimens can strengthen underperforming orofacial muscles, as well as the tongue. Various devices are known to help retrain a person to breathe through the nose instead of the mouth. One such category of devices is known as “mouth tape.” Mouth tape is an adhesive covering worn over a person's mouth that forces, or at least encourages, nasal breathing by either occluding the mouth as an airway or restraining movement of the lips (i.e., out of a closed, or sealed, position).

The drawbacks of known mouth tape products range from mild to severe. Overly restrictive mouth tape may obstruct the wearer's airway to a degree that causes the wearer to struggle to breathe. If adhesion of the tape is too strong, it can cause pain when removed or even damage delicate skin around the lips. The most common side effects are skin irritation, redness, and allergic reactions caused by the adhesives and materials commonly used in mouth tape.

### SUMMARY OF THE INVENTION

The present invention overcomes the drawbacks of existing sleep aid and breath retraining devices by providing a mouth tape that is comfortable to wear, securely affixing to a person's skin but also being easily removable without pain or irritation. The mouth tape features a covering material and an adhesive that are both free of common allergenic materials. The composition of the covering material provides sufficient elasticity to retain a wearer's lips in a sealed position, while also deforming to allow the lips to part in response to intentional movements. An aperture through the covering material allows air to pass through the mouth tape, and may further be functionally sized for the wearer to perform waking activities such as drinking through a straw. A removable paper backing protects the adhesive layer until the wearer is ready to put on the device.

A device in accordance with the present invention may in various embodiments include: a lip-shaped strip of covering material having a thickness of less than one millimeter; and, an adhesive layer disposed on an inner surface of the strip, the adhesive layer comprising an adhesive composition that adheres to skin without irritating the skin. The covering material may be or include a blend of cotton and spandex. The covering material may contain about 95 percent cotton and about five percent spandex. The strip may have a perimeter that includes a series of alternating lobes and valleys, the lobes including first and second lobes that align with the wearer's philtral columns, and the valleys including a first valley disposed between the first and second lobes and aligning with the wearer's philtrum. The lobes May further include a third lobe that aligns with the wearer's left oral commissure and a fourth lobe that aligns with the wearer's right oral commissure, and the valleys may include a second valley between the third lobe and the first lobe, and a third valley between the fourth lobe and the second lobe. The lobes may further include third and fourth lobes that align with non-midline tubercules of the wearer's lower lip. The device may further include an aperture disposed through the strip and the adhesive layer at a location that aligns with the wearer's oral cavity, the aperture having a size that allows a straw to pass through the aperture. The adhesive composition may be a medical-grade acrylic adhesive, such as 44%-46% acrylate copolymer and 54%-56% acetic ether.

### DESCRIPTION OF THE DRAWINGS

FIG. 1 is a front view of an example embodiment of a breath training device in accordance with the present disclosure.

FIG. 2 is a rear view of the example device of FIG. 1.

FIG. 3 is a rear view of the example device of FIG. 1 with the protective paper backing removed.

FIG. 4 is a top view of the example device of FIG. 1 with the protective paper backing removed.

### DETAILED DESCRIPTION

Before any embodiments of the invention are explained in detail, it is to be understood that the invention is not limited in its application to the details of construction and the arrangement of components set forth in the following description or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” “integrated,” and “coupled” and variations thereof are used broadly and encompass both direct and indirect mountings, connections, supports, and couplings. Further, “connected” and “coupled” are not restricted to physical or mechanical connections or couplings. Additional definitions follow.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

The present disclosure provides a device to be worn over a person's mouth and to lightly retain the person's lips in a sealed position, for use as a breath training aid, sleep aid, and snoring reduction aid. To wit, the device when properly worn reduces or eliminates involuntary separation of the lips, thereby discouraging mouth breathing. The device includes a formed strip of covering material and an adhesive layer disposed on an inner surface of the strip. The strip may be “formed” to feature a perimeter shaped to conform to a mouth area of the wearer, the mouth area including the lips, the skin above the top lip and below the bottom lip, and the skin approximate the corners of the mouth, but not extending into regions of the cheeks, chin, or nose. With this shape and commensurate dimensions, the device adheres completely over the lips and makes minimal contact with the skin otherwise. This ergonomic ‘lip-shaped’ design of the strip not only ensures a secure fit but also, in cooperation with the material of the strip, helps retain moisture and hydration when used with lip and skin care products, enhancing comfort and preventing excessive dryness, a common issue with traditional mouth tapes.

An aperture may be formed in the strip in alignment with the oral aperture (i.e., mouth opening) to allow for intentional separation of the lips, for breathing or functional tasks such as drinking through a straw. The covering material may be moderately elastic and breathable, so the device is comfortable to wear. The covering material may be a medical-grade material that is free of common allergenic substances such as latex and silicone-containing compositions. Similarly, the adhesive, which is not water soluble but can be removed with soap and water, has a composition that is non-allergenic and gentle on skin. Further, the adhesive composition is compatible with skincare products such as night creams, lip serums, and masks; this ensures that the device stays in place while allowing users to maintain their skincare routines without compromising adhesion or effectiveness. A paper or disposable plastic backing may be attached to the adhesive layer to protect the adhesive until the backing is removed to apply the device.

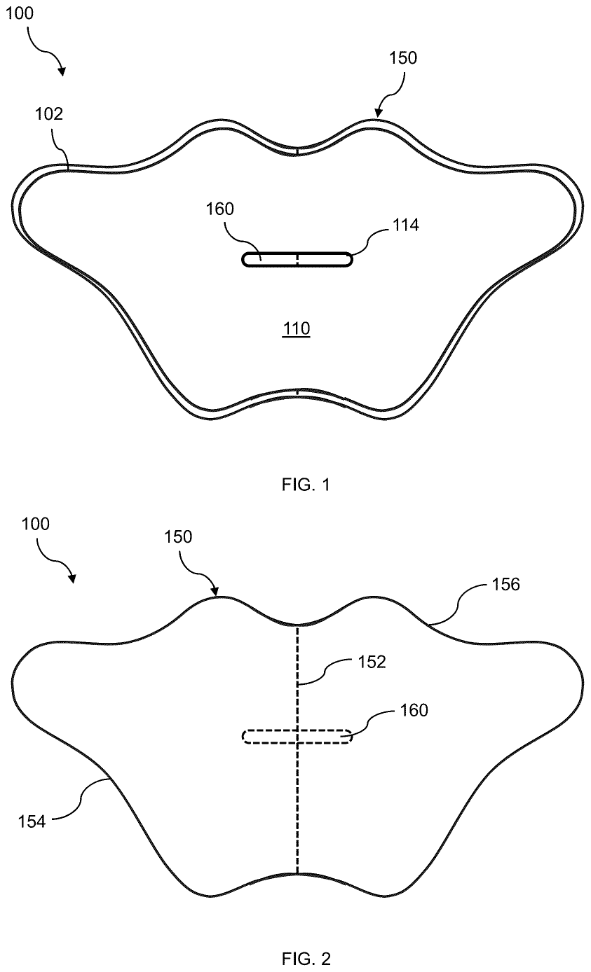

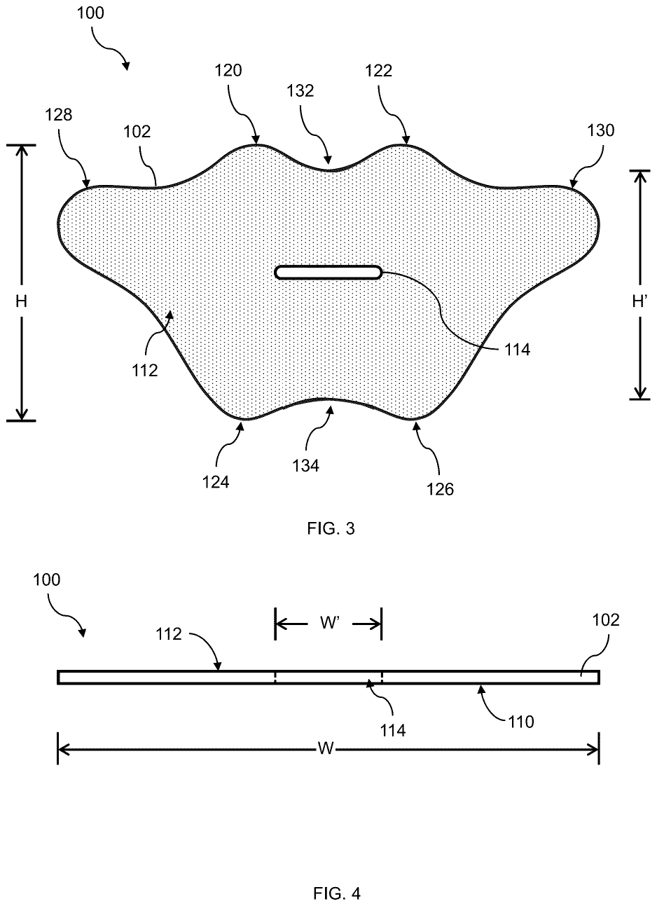

Referring to FIGS. 1-4, an example embodiment is shown of a device 100 that, in accordance with the present disclosure, can be affixed to a person's face over the mouth area when the person's lips are sealed, and the device 100 resists involuntary movements to retain the lips in the sealed position. In various embodiments, the device 100 is a strip 102 of medical-grade covering material with an outer surface 110 and an opposing inner surface (not numbered) that supports an adhesive layer 112. Advantageously for the application of the device 100 as a breath training and sleep aid that adheres to areas of highly sensitive skin, the compositions of the covering material in the strip 102 and the adhesive in the adhesive layer 112 are selected to minimize the risks of allergic reactions, skin irritation, comedone genesis, and over-adhesion resulting in pain removing the device 100. Examples of suitable covering materials include medical-grade gauzes and tapes that do not contain silicone or latex. Additional desirable features of the covering material are: moderate elasticity, so the strip deforms but remains adhered when the wearer intentionally moves the lips (e.g., to speak, to breathe, or to drink from a straw); and, breathability, to reduce claustrophobic feelings and otherwise improve comfort; sweat resistance; and, tear resistance. One particular example composition of the covering material is a blend of cotton and spandex, specifically 90%-95% cotton and 5%-10% spandex. A suitable thickness of the example breathable covering material for the strip 102 is between about 0.5 mm and 1 mm, and preferably 0.6 mm.

The adhesive must provide the desired skin sensitivity while also being compatible with the covering material; the adhesive also should resist solubility in water due to contact with the skin, water-based skincare products, and the wearer's exhalations. For example, the adhesive provides gentle adhesion to the skin even when placed in contact over commonly-used night creams, night masks, lip serums, and other typical topical compositions used in skincare regimens. Various medical-grade adhesive compositions are suitable, including surgical glues and acrylic adhesives provided they do not contain harsh solvents or irritants. An example adhesive composition for use with a cotton-spandex blend covering material is an acrylic adhesive comprising 44%-46% acrylate copolymer and 54%-56% acetic ether. The adhesive layer 112 may cover the entire inner surface of the strip 102, or substantially all (i.e., 90% or 95% or more) of the inner surface, in various embodiments.

The strip 102 may be cut, molded, spun, or otherwise formed to have a size and a perimeter shape that contacts as little skin surface area as needed to establish secure adhesion of the device 100 over the mouth when the lips are sealed. Noting that the faces, mouths, and lips of potential wearers of the device 100 come in all shapes and sizes, the device 100 may be made in a variety of dimensional combinations; the dimensions of the device 100 have been designed to accommodate a diverse range of mouth sizes and lip structures. By offering an adaptable shape and optimal flexibility, the device 100 ensures a comfortable fit for different facial anatomies, making it inclusive for a wide variety of users. In some embodiments, for the sake of mass production, a set of dimensions may be selected to fit the broadest swath of adults (or, alternatively, children) having typical lip fullness and eversion. In one embodiment, the planar maximum dimensions (i.e., width W and height H, measured between extreme edges) of the strip 102 may be 3.5 inches to 4.5 inches, and preferably four inches, in width W, and 1.75 inches to 2.5 inches, and preferably 2 inches, in height H.

The perimeter shape of the strip 102, referred to herein as “mouth shape” or “lip shape,” has an undulating pattern comprising alternating convex sections, or “lobes,” and concave sections, or “valleys,” that conform to anatomical structures within a “mouth area” of the human face. The ergonomic “mouth shape” or “lip shape” design of the strip 102 not only ensures a secure fit but also helps retain moisture and hydration when used with lip and skin care products. This feature enhances comfort and prevents excessive dryness, a common issue with traditional mouth tapes. A first lobe 120 and a second lobe 122 formed in the top edge of the strip 102 align substantially with the “cupid's bow” tubercules of the upper lip; these lobes 120, 122 contact and are adhered to the skin of the philtral columns, but preferably do not enter the nasal area. A first valley 132 is formed in the top edge of the strip 102 between the first lobe 120 and second lobe 122, aligning with the philtrum but minimally contacting it approximate the midline of the upper lip. A third lobe 124 and a fourth lobe 126 formed in the bottom edge of the strip 102 align substantially with the (non-midline) tubercules of the lower lip, which are more prominent in some individuals than others; these lobes 124, 126 contact and are adhered to the perioral skin below the lower lip, but preferably do not extend past, into, or to the labiomental groove. A second valley 134 is formed in the bottom edge of the strip 102 between the third lobe 124 and fourth lobe 126 approximate the midline of the lower lip. An inner height H′ can be measured between the apexes of the first valley 132 and second valley 134, which apexes are aligned on the midline of the strip 102. In an embodiment where the maximum height H measured at opposing lobes adjacent to the valleys 132, 134 is approximately 2 inches, the inner height H′ of the strip 102 at the midline may be about 1.5 inches.

Corresponding to the corners of the mouth, the perimeter of the strip 102 projects at its left and right edges to form elongated lobes 128, 130. A fifth lobe 128 may contact the perioral skin approximate the left corner of the wearer's mouth, covering the left oral commissure but preferably not extending into the left buccal region of the face. Similarly, a sixth lobe 130 may contact the perioral skin approximate the right corner of the wearer's mouth, covering the right oral commissure but preferably not extending into the right buccal region of the face. Thus, the preferred four-inch width measured between the apexes of the fifth lobe 128 and sixth lobe 130 provides perioral skin contact and adhesion substantially or entirely within the oral region of the face. Preferably, the strip 102 does not intersect or cross the nasolabial folds, which might crease the strip 102 and cause the device 100 to loosen. The edges of the strip 102 defining its perimeter may have additional lobes, as well as valleys (not labeled) between adjacent lobes (e.g., between the first lobe 120 and the fifth lobe 128; between the fourth lobe 126 and the sixth lobe 130). The valleys provide concave curvature of the strip 102 edges to reduce the amount of contact and adhesion between the device 100 and the skin, compared to straight edges between the apexes of adjacent lobes, in areas where contact is not necessary for the device 100 to adequately maintain the lips in the sealed position.

In some embodiments, an aperture 114 may be formed through the strip 102 at a location that aligns with a portion of the oral aperture (i.e., the opening of the mouth, between parted lips). The aperture 114 ensures that the device 100 does not completely obstruct the mouth as an airway, and as a route to the stomach. The device 100 may be worn during waking periods, for the purpose of training the wearer in nasal breathing; the aperture 114 allows for intentional parting of the lips for functional access, such as inserting a straw through the aperture 114 for drinking. Additionally, the aperture 114 allows the wearer to draw breath through the device 100 intentionally or in emergent circumstances. In some embodiments, the aperture 114 may be approximately centered in the surface area of the strip 102. The aperture 114 may have a width W′ that best facilitates access to the mouth and oral cavity without compromising the resilience of the strip 102 in its ability to hold the lips in the sealed position. For example, the width W′ of the aperture 114 may be about 0.5 inches to 0.75 inches.

As illustrated, in some embodiments the adhesive layer 112 may be protected with a paper backing layer 150. The paper backing layer 150 may be a coated (e.g., via wax coating) or uncoated paper or plastic layer that sticks to the adhesive but does not degrade the adhesiveness of the adhesive. The paper backing layer 150 may cover the entirety of the adhesive layer 112. In some embodiments, the paper backing layer 150 may comprise a first section 154 and a second section 156 separated by a perforation 152 that allows the sections 154, 156 to be torn apart for easy removal from the device 110. In some embodiments, the paper backing layer 150 may further include a portion 160 that covers the aperture 114 and is removable from the other sections 154, 156.

Therefore, in one aspect, the present disclosure provides a mouth tape including a lip-shaped strip of covering material having a thickness of less than one millimeter, and an adhesive layer disposed on an inner surface of the strip, the adhesive layer comprising an adhesive composition that adheres to skin without irritating the skin. The covering material may be or include a blend of cotton and spandex. The covering material may contain about 95 percent cotton and about five percent spandex. The strip may have a perimeter including a series of alternating lobes and valleys, the lobes including first and second lobes that align with the wearer's philtral columns, and the valleys including a first valley disposed between the first and second lobes and aligning with the wearer's philtrum. The lobes may further include a third lobe that aligns with the wearer's left oral commissure and a fourth lobe that aligns with the wearer's right oral commissure, and the valleys may further include a second valley between the third lobe and the first lobe, and a third valley between the fourth lobe and the second lobe. The lobes may further include third and fourth lobes that align with non-midline tubercules of the wearer's lower lip.

The mouth tape may further include an aperture disposed through the strip and the adhesive layer at a location that aligns with the wearer's oral cavity, the aperture having a size that allows a straw to pass through the aperture. The adhesive composition may be a medical-grade acrylic adhesive. The adhesive composition may contain about 44%-46% acrylate copolymer and 54%-56% acetic ether.

The inventions and methods described herein can be viewed as a whole, or as a number of separate inventions, that can be used independently or mixed and matched as desired. All inventions, steps, processes, devices, and methods described herein can be mixed and matched as desired. All previously described features, functions, or inventions described herein or by reference may be mixed and matched as desired. It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

What is claimed is:

1. A mouth tape comprising:
a strip of covering material having a thickness of less than one millimeter and comprising a blend of cotton and spandex; and
an adhesive layer disposed on an inner surface of the strip, the adhesive layer comprising an adhesive composition that adheres to skin without irritating the skin.

2. The mouth tape of claim 1, wherein the covering material comprises about 95 percent cotton and about five percent spandex.

3. The mouth tape of claim 2, wherein the strip is “lip-shaped” in that the strip has a perimeter and size that covers a wearer's upper and lower lips and the wearer's oral commissures and does not contact the wearer's cheek areas, nasal area, or chin area.

4. The mouth tape of claim 3, wherein the perimeter of the strip includes a series of alternating lobes and valleys, the lobes including first and second lobes that align with the wearer's philtral columns, and the valleys including a first valley disposed between the first and second lobes and aligning with the wearer's philtrum.

5. The mouth tape of claim 3, further comprising an aperture disposed through the strip and the adhesive layer at a location that aligns with the wearer's oral cavity, the aperture having a size that allows a straw to pass through the aperture.

6. The mouth tape of claim 3, wherein the adhesive composition comprises about 44%-46% acrylate copolymer and 54%-56% acetic ether.

7. A mouth tape comprising:
a lip-shaped strip of covering material having a thickness of less than one millimeter; and
an adhesive layer disposed on an inner surface of the strip, the adhesive layer comprising an adhesive composition that adheres to skin without irritating the skin.

8. The mouth tape of claim 7, wherein the covering material comprises a blend of cotton and spandex.

9. The mouth tape of claim 8, wherein the covering material comprises about 95 percent cotton and about five percent spandex.

10. The mouth tape of claim 7, wherein the strip has a perimeter comprising a series of alternating lobes and valleys, the lobes including first and second lobes that align with the wearer's philtral columns, and the valleys including a first valley disposed between the first and second lobes and aligning with the wearer's philtrum.

11. The mouth tape of claim 10, wherein the lobes further include a third lobe that aligns with the wearer's left oral commissure and a fourth lobe that aligns with the wearer's right oral commissure, and wherein the valleys further include a second valley between the third lobe and the first lobe, and a third valley between the fourth lobe and the second lobe.

12. The mouth tape of claim 10, wherein the lobes further include third and fourth lobes that align with non-midline tubercules of the wearer's lower lip.

13. The mouth tape of claim 7, further comprising an aperture disposed through the strip and the adhesive layer at a location that aligns with the wearer's oral cavity, the aperture having a size that allows a straw to pass through the aperture.

14. The mouth tape of claim 7, wherein the adhesive composition comprises a medical-grade acrylic adhesive.

15. The mouth tape of claim 14, wherein the adhesive composition comprises about 44%-46% acrylate copolymer and 54%-56% acetic ether.
